(19) United States
(12) Patent Application Publication
(10) Pub. No.: US 2007/0129625 A1
(43) Pub. Date: Jun. 7, 2007

(54) Systems and methods for detecting the presence of abnormalities in a medical image

(75) Inventors: Wenguang Li, Campbell, CA (US); Shashidhar Sathyanarayana, Pleasanton, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: Boston Scientific Scimed Systems, Inc.

(21) Appl. No.: 11/285,692

(22) Filed: Nov. 21, 2005

(51) Int. Cl. A61B 5/05; A61B 8/00; A61B 6/00

(52) U.S. Cl. 600/407; 600/476; 600/437

(57) ABSTRACT

The invention is directed to systems and methods for detecting and presenting textural information from medical images. In one example embodiment, a medical imaging system includes an imaging transducer assembly configured to emit one or more energy pulses and receive one or more echo signals, and a console, coupled to the imaging transducer assembly, configured to receive the one or more echo signals, detect one or more signals that correspond with an abnormality, and invoke an alert in response to the detection of the one or more signals that correspond with an abnormality.

### FIELD OF THE INVENTION

The field of the invention relates to medical imaging systems, and more particularly to systems and methods for detecting and alerting an operating of the presence of abnormalities within a medical image.

### BACKGROUND OF THE INVENTION

Intraluminal, intracavity, intravascular, and intracardiac treatments and diagnosis of medical conditions utilizing minimally invasive procedures are effective tools in many areas of medical practice. These procedures are typically performed using imaging and treatment catheters that are inserted percutaneously into the body and into an accessible vessel of the vascular system at a site remote from the vessel or organ to be diagnosed and/or treated, such as the femoral artery. The catheter is then advanced through the vessels of the vascular system to the region of the body to be treated. The catheter may be equipped with an imaging device, typically an ultrasound imaging device, which is used to locate and diagnose a diseased portion of the body, such as a stenosed region of an artery. For example, U.S. Pat. No. 5,368,035, issued to Hamm et al., the disclosure of which is incorporated herein by reference, describes a catheter having an intravascular ultrasound imaging transducer.

FIG. 1a shows an example of an imaging transducer assembly 1 known in the art. The imaging transducer 1 is typically within the lumen 10 of a guidewire (partially shown), having an outer tubular wall member 5. To obtain an image of a blood vessel, the imaging transducer assembly 1 may be inserted into the vessel. The transducer assembly 1 may then rotate while simultaneously emitting energy pulses, e.g., ultrasound waves, at portions of the vessel from within the vessel and receiving echo or reflected signals.

Turning to FIG. 1b, it is known in the art that an imaging console 20 having a display screen, a processor and associated graphics hardware (not shown) may be coupled with the imaging transducer assembly 1 to form a medical imaging system 30. The imaging console 20 processes the received echo signals from the imaging transducer assembly 1 and forms images of the area being imaged. To form the images, the imaging console 20 draws multiple lines, known as “radial lines”, (not shown) on the display screen that each correspond to an angular position of the transducer assembly 1. The processor of the imaging console 20 assigns brightness values to pixels of the lines based on magnitude levels of the echo signals received from the transducer assembly 1 at the angular positions corresponding to the lines. A drawing that includes a large number of these radial lines results in an image such as an intravascular ultrasound (IVUS) image (not shown). Such an image may show, among other things, the texture of the area being imaged, such as the smoothness or the roughness of the surface of the area being imaged.

An example of an image 70 having a large range of magnitudes and a number of texturally distinct regions 80 is shown in FIG. 1c. Texture and the correct discrimination of the underlying surface are important in medical imaging. Such information is helpful to radiologists and other clinicians who seek to diagnose pathology. It is often the case in medical imagery that an abnormality is detectable only as a subtle variation in texture.

Accordingly, an improved system and method for detecting and presenting such textural information would be desirable.

### SUMMARY OF THE INVENTION

The invention is directed to systems and methods for detecting and presenting textural information from medical images. In one example embodiment, a medical imaging system includes an imaging transducer assembly configured to emit one or more energy pulses and receive one or more echo signals, and a console, coupled to the imaging transducer assembly, configured to receive the one or more echo signals, detect one or more signals that correspond with an abnormality, and invoke an alert in response to the detection of the one or more signals that correspond with an abnormality.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

In order to better appreciate how the above-recited and other advantages and objects of the inventions are obtained, a more particular description of the embodiments briefly described above will be rendered by reference to specific embodiments thereof, which are illustrated in the accompanying drawings. It should be noted that the components in the figures are not necessarily to scale, emphasis instead being placed upon illustrating the principles of the invention. Moreover, in the figures, like reference numerals designate corresponding parts throughout the different views. However, like parts do not always have like reference numerals. Moreover, all illustrations are intended to convey concepts, where relative sizes, shapes and other detailed attributes may be illustrated schematically rather than literally or precisely.

FIG. 1a is a cross-sectional side view of an imaging transducer assembly known in the art.

FIG. 1b is a block diagram of a medical imaging system known in the art.

FIG. 1c is an example of an image showing different magnitudes and textures.

FIG. 2 is an example medical image of vulnerable coronary plaque.

FIG. 3a is a diagram of the operation of a preferred example embodiment of the invention.

FIG. 3b is a diagram of the operation of a preferred example embodiment of the invention.

### DETAILED DESCRIPTION OF THE PREFERRED EMBODIMENTS

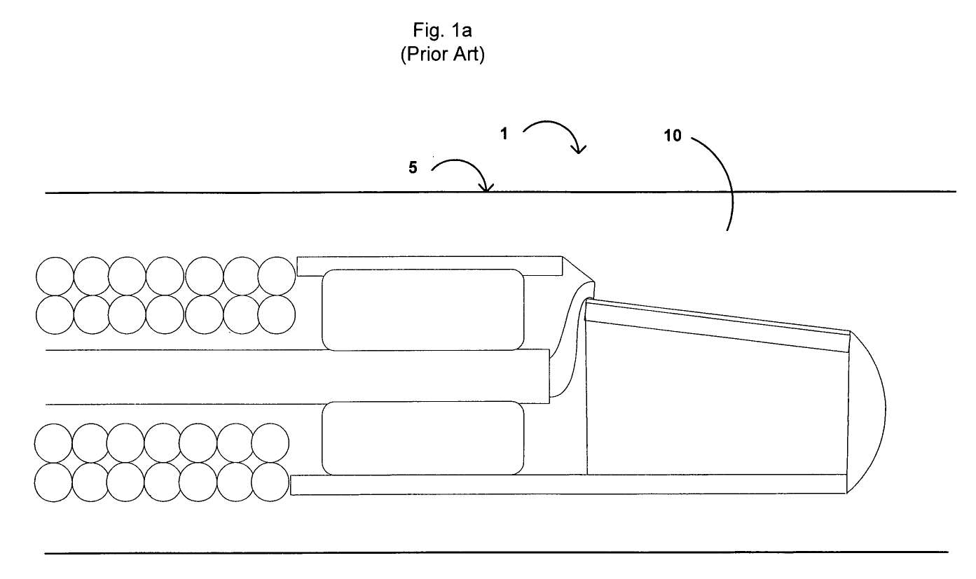

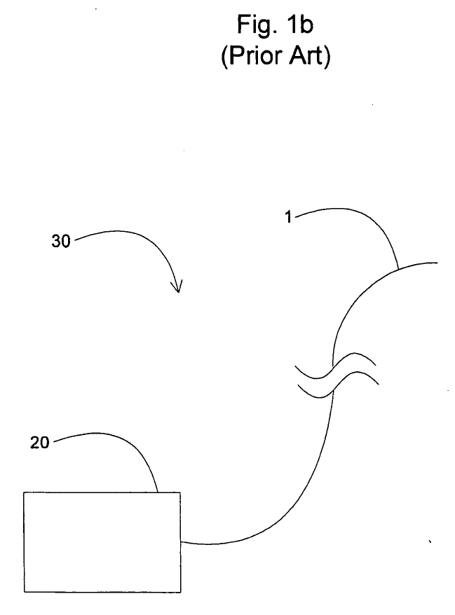

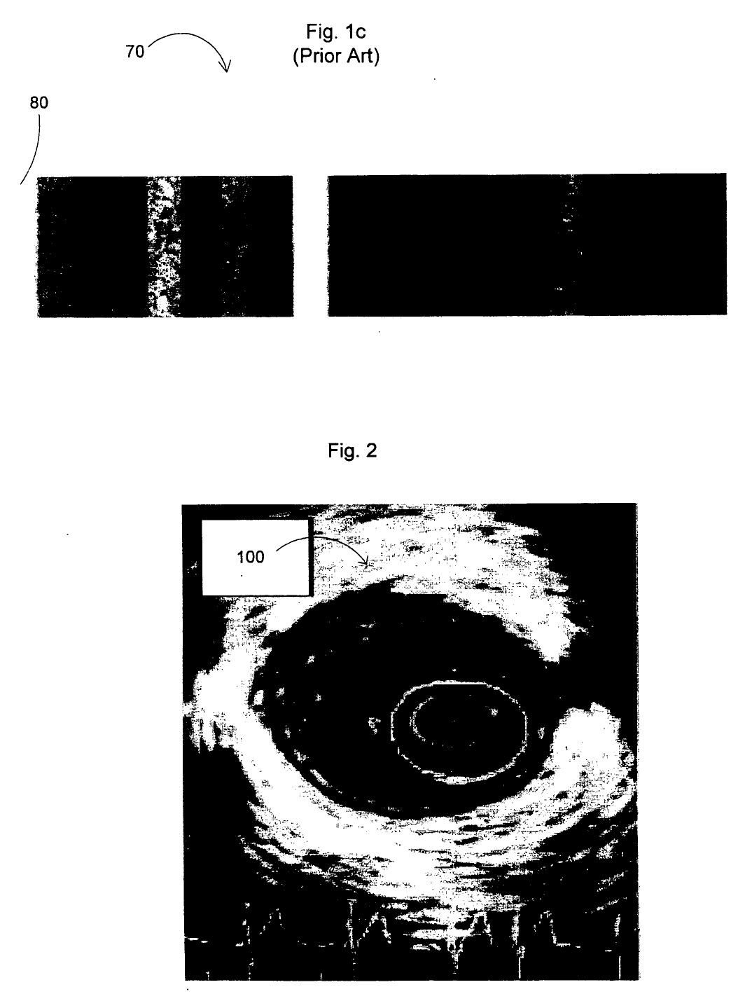

Turning to FIG. 1b, a typical imaging system 30 may include an imaging transducer assembly 1 and coupled to the imaging transducer assembly 1, an imaging console 20 having a display screen, a processor and associated graphics hardware (not shown). To form an image of body tissue by an intravascular ultrasound system (IVUS), the imaging transducer assembly 1 emits energy pulses, such as ultrasound pulses, and receives echo signals from those pulses after they are reflected by body tissue (tissue, fat, bone, vessel, plaque, etc., or other object). If desired, the imaging transducer may emit energy pulses while simultaneously rotating about a central axis or translate longitudinally along the central axis. The imaging console 20 receives the echo signals from the imaging transducer assembly 1 and draws lines on the display screen that each correspond to an angular position of the transducer assembly 1 as the transducer assembly 1 rotates. The processor of the imaging console 20 assigns brightness values to pixels of the lines based on the magnitude levels of echo signals received from the transducer assembly 1 at the corresponding angular positions. The echo signals received are typically classified by records, or vectors, corresponding to a particular angular position. Each record, or vector, for a particular angular position contains oscillations covering a large range of magnitudes. A drawing that includes a large number of these vectors or lines (“radial lines”) results in an image, such as an IVUS image, shown in FIG. 2. An example of an image 70, which may be an image of received echo signals, containing a large range of magnitudes and a number of texturally distinct regions 80 is shown in FIG. 1c.

An IVUS image may provide textural information about the area being imaged, such as the appearance of blood speckle. Further, such images can provide tissue characterization and information about the existence of a variety of abnormalities within the area being imaged. One example abnormality that can be detected from an IVUS image is vulnerable plaque, which refers to a subgroup of often modestly stenoic plaques that are prone to rupture or erosion. An accumulation of such plaque within an artery can result in acute coronary syndromes and sudden cardiac death. FIG. 2 illustrates an example IVUS image of an artery having vulnerable coronary plaque 100. Such abnormalities can be identified in an IVUS image by a trained technician. Further, there are computer programs known in the art having algorithms that recognize such abnormalities. However, because a human body's vascular system is so complicated and because there are an indeterminate number of characteristics that a technician searches for in a medical image, the operation of an imaging system can be unwieldy.

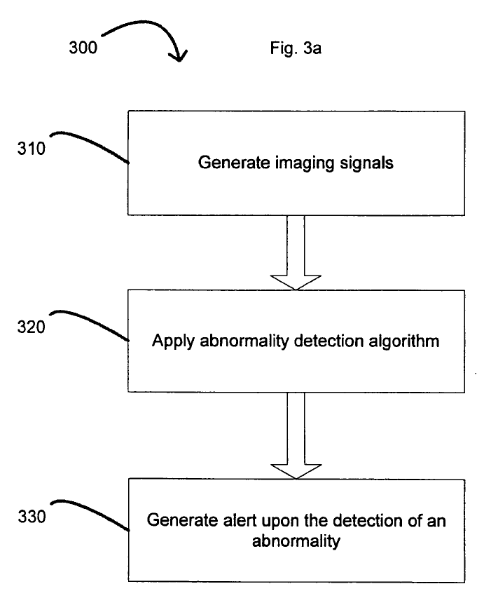

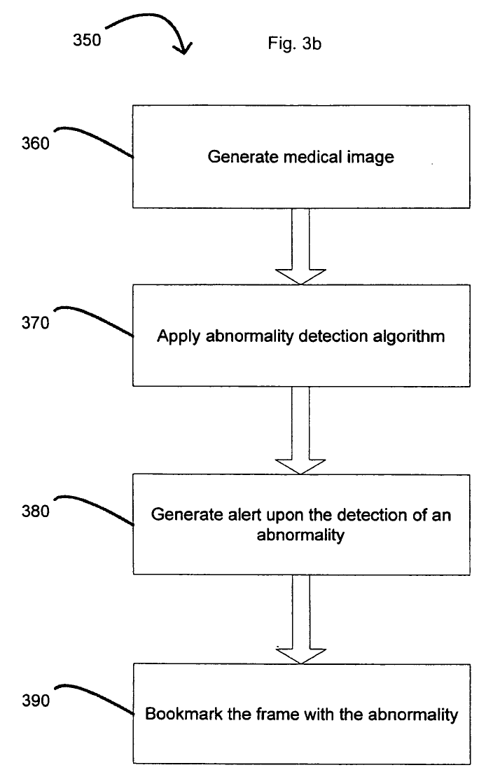

One approach to alleviate and simplify the burden of operating a medical imaging system 30 is shown in FIG. 3a, which illustrates the operation 300 of an alert system within a medical imaging system 30. The alert system is preferably implemented as a computer program within the console 20. The medical imaging system 30 first generates a medical image signal, such as a radio frequency ultrasound signal in real-time or near real-time (action block 310). One or more algorithms are then applied to the signals in search for particular abnormalities, such as vulnerable plaque (action block 320). Such algorithms generally include searching for particular patterns within the image that corresponds with particular types of tissue or vessel morphology known in the art. For example, techniques for detecting vulnerable plaque using a medical imaging system, such as IVUS imaging system or an Optical Coherence Tomography (“OCT”) system, are described in Briain D. MacNeill, Harry C. Lowe, Masamichi Takano, Valentin Fuster,and Ik-Kyung Jang, Intravascular Modalities for Detection of Vulnerable Plaque Current Status, ARTERIOSCLER THROMB VASC. BIOL., August 2003, p. 1333 (available at http://www.atvbaha.org); and Andrew L. McCleod, Robin J. Watson, Thomas Anderson, Scott Inglis, David E. Newby, David B. Northridge, Neal G. Uren, and W. N. McDicken, Classification of Arterial Plaque by Spectral Analysis in Remodelled Human Atherosclerotic Coronary Arteries, ULTRASOUND IN MED. & BIOL., Vol. 30, No. 2, pp. 155-159, 2004., which are hereby incorporated by reference. These references disclose methods of detecting vulnerable plaque by searching for characteristics such as a thin fibrous cap, a large lipid-rich pool, and/or increased macrophage activity, utilizing techniques such as integrated backscattering and spectral analysis, as one of ordinary skill in the art will appreciate. Upon the detection of a particular abnormality, an alert system can be invoked (action block 330). The alert system may include an audio and/or visual alarm, such as a flashing symbol on the screen of the imaging console 20. Specific types of alarms, audio and/or visual, can be assigned to specific types of abnormalities. For example, an alarm for vulnerable plaque can announce “vulnerable plaque” or provide an audio and/or visual representation of vulnerable plaque.

In an alternative approach 350, instead of, or in addition to, analyzing the medical imaging signals, analysis can be performed on a processed image, as illustrated in FIG. 3b. First, a medical image, such as an IVUS or OCT image is generated (action block 360). Then, the algorithms described above may be applied to the image (action block 370), and a visual and/or audio alarm can be invoked in response to the detection of an abnormality, as described above (action block 380). For imaging systems that generate images in frames, the alert system can enable an operator to bookmark frames, or retain frames, in which the presence of an abnormality is suspected (action block 390).

These approaches can be applied to imaging catheter coupled to automatic pull-back systems (not shown) or manual pullback systems (not shown). An imaging system having an imaging catheter coupled to an automatic pull-back system is disclosed in U.S. Pat. No. 5,799,655, to Jang, et al., which is hereby incorporated by reference in its entirety.

As will be appreciated by one of ordinary skill in the art, laboratories that conduct IVUS imaging often require high workflow efficiency. Because a vascular system is a complicated structure and because an innumerable amount of abnormalities may exist, the alert systems described above may facilitate the desirable workflow efficiency by allowing physicians to quickly determine which portions of the vascular system require a more detailed examination based on alerts, which function as trigger points. Thus, it is desirable that the detection algorithms described above function with a higher sensitivity rather than specificity.

In the foregoing specification, the invention has been described with reference to specific embodiments thereof. It will, however, be evident that various modifications and changes may be made thereto without departing from the broader spirit and scope of the invention. For example, the reader is to understand that the specific ordering and combination of process actions described herein is merely illustrative, and the invention can be performed using different or additional process actions, or a different combination or ordering of process actions. For example, this invention is particularly suited for applications involving medical imaging devices, but can be used on any design involving imaging devices in general. As a further example, each feature of one embodiment can be mixed and matched with other features shown in other embodiments. Additionally and obviously, features may be added or subtracted as desired. Accordingly, the invention is not to be restricted except in light of the attached claims and their equivalents.

What is claimed is:

1. A medical imaging system comprising:
an imaging transducer assembly configured to emit one or more energy pulses and receive one or more echo signals; and
a computer console coupled to the imaging transducer assembly and configured to receive the one or more echo signals, detect one or more signals that correspond with an abnormality, and invoke an alert in response to the detection of the one or more signals that correspond with an abnormality.

2. The medical imaging system of claim 1, wherein the abnormality is vulnerable plaque.

3. The medical imaging system of claim 1, wherein the alert is at least one of an audio alert and a visual alert.

4. The medical imaging system of claim 1, wherein the console is configured to detect a plurality of abnormalities, and assign a unique alert to each of the plurality of abnormalities.

5. The medical imaging system of claim 1, wherein the console is configured to detect a plurality of abnormalities, and assign a unique alert to at least two of the plurality of abnormalities.

6. The medical imaging system of claim 1, wherein the imaging transducer assembly has an axis and is configured to rotate on its axis, and wherein the imaging transducer assembly emits energy pulses and receives one or more echo signals while rotating on its axis.

7. The medical imaging system of claim 1, wherein the imaging transducer assembly is an ultrasound transducer assembly.

8. The medical imaging system of claim 1, wherein the imaging transducer assembly is an optical coherence tomography device.

9. The medical imaging system of claim 1, wherein the computer console includes a processor, a display screen, and graphics hardware.

10. The medical imaging system of claim 1, further comprising an automatic pullback system coupled to the imaging transducer assembly.

11. The medical imaging system of claim 1, wherein the computer console is configured to generate a plurality of frames of medical images, and configured to enable a user to bookmark a frame in which an abnormality is detected.

12. A method for imaging a vascular system comprising the steps of:
inserting an imaging catheter into the vascular system;
generating one or more image signals via the imaging catheter;
analyzing the one or more image signals in real-time for an abnormality; and
invoking an alert once an abnormality is detected.

13. The method of claim 12, wherein the abnormality is vulnerable plaque.

14. The method of claim 12, wherein the alert is at least one of an audio alert and a visual alert.

15. The method of claim 12, further comprising analyzing the one or more image signals in real-time for a plurality of abnormalities, and assigning a plurality of unique alerts, each corresponding with one of the plurality of abnormalities.

16. The method of claim 12, wherein the image signals are generated as the imaging catheter is automatically pulled back.

17. The method of claim 12, further comprising generating a plurality of frames of medical images and enabling a user to bookmark a frame in which an abnormality is detected.

18. A medical imaging system comprising:
an imaging transducer assembly configured to emit one or more energy pulses and receive one or more echo signals; and
a computer console coupled to the imaging transducer assembly having a processor and a computer readable medium with a set of instructions to be executed by the processor, said instructions including:
receiving the one or more echo signals,
detecting one or more signals that correspond with an abnormality,
assigning a unique alert with the abnormality; and
and invoking the unique alert in response to the detection of the one or more signals that correspond with the abnormality.

19. The medical imaging system of claim 18, wherein the abnormality is vulnerable plaque.

20. The medical imaging system of claim 18, wherein the alert is at least one of an audio alert and a visual alert.

21. The medical imaging system of claim 18, wherein the set of instructions further includes detecting a plurality of abnormalities, and assigning a unique alert to each of the plurality of abnormalities.

22. The medical imaging system of claim 18, wherein the set of instructions further includes detecting a plurality of abnormalities, and assigning a unique alert to at least two of the plurality of abnormalities.

23. The medical imaging system of claim 18, wherein the imaging transducer assembly has an axis and is configured to rotate on its axis, and wherein the imaging transducer assembly emits energy pulses and receives one or more echo signals while rotating on its axis.

24. The medical imaging system of claim 18, wherein the imaging transducer assembly is an ultrasound transducer assembly.

25. The medical imaging system of claim 18, wherein the imaging transducer assembly is an optical coherence tomography device.

26. The medical imaging system of claim 18, wherein the computer console includes a processor, a display screen, and graphics hardware.

27. The medical imaging system of claim 18, further comprising an automatic pullback system coupled to the imaging transducer assembly.

28. The medical imaging system of claim 18, wherein the computer console is configured to generate a plurality of frames of medical images, and configured to enable a user to bookmark a frame in which an abnormality is detected.
